(19) United States
(12) Patent Application Publication
(10) Pub. No.: US 2026/0092299 A1
(43) Pub. Date: Apr. 2, 2026

(54) METHODS, SYSTEMS AND DEVICES FOR SCREENING COMPOUNDS FOR EFFICACY AGAINST BIOFILM FORMATION

(71) Applicant: Battelle Memorial Institute, Columbus, OH (US)

(72) Inventors: Fadime KARA MURDOCH, Upper Arlington, OH (US); Jonathan PICKING, Hilliard, OH (US); Angel RUBIO MARRERO, Columbus, OH (US); Sherry BLIGHT, Lewis Center, OH (US)

(73) Assignee: BATTELLE MEMORIAL INSTITUTE, Columbus, OH (US)

(21) Appl. No.: 19/344,186

(22) Filed: Sep. 29, 2025

Related U.S. Application Data
(60) Provisional application No. 63/701,406, filed on Sep. 30, 2024.

(51) Int. Cl. C12Q 1/18

(52) U.S. Cl. CPC C12Q 1/18

(57) ABSTRACT

Disclosed herein are methods, systems and devices for screening compounds for efficacy against biofilm formation. These methods have the advantage over the prior art, in that the biofilm formation (or inhibition thereof) is tested directly on the material of interest, thereby providing a high throughput system for testing antimicrobial formulations against corrosion. Also disclosed is a peg assembly, and kits comprising a peg assembly, wherein the pegs are composed of a material of interest.

### FIELD OF INVENTION

The present disclosure relates to methods, systems and devices for screening efficacy of formulations and compounds against biofilm formation.

### BACKGROUND

Metals and metal alloys are primary materials for the aircraft industry and maritime industry, specifically for aircraft fuel systems, engine components, ship hulls and so on. The presence of the alloying elements in these metals makes them susceptible to localized corrosion at the same time vulnerable to microbial attachment. For example, it is common that certain issues with aircraft deterioration are related to microbial growth by contamination inside fuel storage tanks and aircraft wing tanks; this phenomenon is known as microbiologically influenced corrosion (MIC), caused by biofilm formation on surfaces aircraft or ship parts. As expected, corrosion and biocorrosion increase maintenance costs and time of the aircraft in the hangar.

Current remedies against microbial growth are labor intensive, often impractical, expensive, and can negatively impact human health and the environment. Superior methods of microbial control are urgently needed. Novel antimicrobial coatings with antimicrobial compounds (AMCs) could solve the problem effectively with less labor, a lower required dose and minimal environmental impact. AMCs have been highlighted as promising candidates for antimicrobial surface elaboration due to their biocompatibility, low toxicity, and effectiveness.

One commonly used approach for the evaluation of biofilms on the surface of materials of interest is to incubate coupons of the chosen material in liquid media. Such coupons require individual manipulation by hand, however, and this approach is not conducive to high throughput testing. Other commercialized testing means include Minimum Biofilm Eradication Concentration (MBEC) assay kits. These kits include peg containing plates which are made of polymers like polystyrene. This does not allow for the option of testing biofilm inhibition directly on the surfaces of interest, such as aluminum or aluminum alloys.

In light of the current drawbacks, there remains a need for high throughput means for MBEC assays directly on the material of interest. There remains a need to support the screening of novel AMCs and development of anti-biofilm coatings and formulations. It is the object of this invention to provide a high-throughput laboratory screening system that would enable broad screens and/or a range of AMC concentrations with many replicates.

### SUMMARY

Disclosed herein are methods, systems and devices for screening compounds for efficacy against biofilm formation. These methods have the advantage over the prior art, in that the biofilm formation (or inhibition thereof) is tested directly on the material of interest, thereby providing a high throughput system for testing antimicrobial formulations against corrosion or other consequences of biofilm formation on surfaces of interest.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all subcombinations of the various embodiments and elements thereof are also specifically embraced by the present invention and are disclosed herein just as if each and every such subcombination was individually and explicitly disclosed herein.

### SELECTED DEFINITIONS AND NOMENCLATURE

As used herein the term “materials of interest” refers to materials which are directly being tested upon to investigate biofilm formation and/or effect thereof. It does not refer to common laboratory assay kit materials, such as commonly used polymers for labware.

The singular forms “a”, “an” and “the” are intended to include the plural forms as well, unless the context clearly indicates otherwise.

The term “and/or” or “and or” refers to and encompasses any and all possible combinations of one or more of the associated listed items.

The term “about,” when referring to a measurable value such as length, width, diameter, radius, or an amount of a compound, dose, time, temperature, and the like, is meant to encompass variations of 10%, 5%, 1%, 0.5%, or even 0.1% of the specified amount.

In the interest of brevity and conciseness, any ranges of values set forth in this specification contemplate all values within the range and are to be construed as support for claims reciting any sub-ranges having endpoints which are real number values within the specified range in question. By way of a hypothetical illustrative example, a disclosure in this specification of a range of from 1 to 5 shall be considered to support claims to any of the following ranges: 1-5; 1-4; 1-3; 1-2; 2-5; 2-4; 2-3; 3-5; 3-4; and 4-5.

The term “substantially” is utilized herein to represent the inherent degree of uncertainty that can be attributed to any quantitative comparison, value, measurement, or other representation. The term “substantially” is also utilized herein to represent the degree by which a quantitative representation can vary from a stated reference without resulting in a change in the basic function of the subject matter at issue.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

The terms “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the present disclosure.

As used throughout this description, and in the claims, a list of items joined by the term “at least one of” or “one or more of” can mean any combination of the listed terms. For example, the phrase “at least one of X, Y or Z” can mean X; Y; Z; X and Y; X and Z; Y and Z; or X, Y and Z.

All patents, patent applications and publications referred to herein are incorporated by reference in their entirety.

### BRIEF DESCRIPTION OF THE DRAWINGS

Other advantages of the present invention will be apparent from the following detailed description of the invention in conjunction with embodiments as illustrated in the accompanying drawings, in which:

FIG. 1 depicts a peg assembly in accordance with embodiments disclosed herein.

FIGS. 2A-2C depict various views of a peg assembly in accordance with embodiments disclosed herein.

FIG. 3 depicts a graphic of screening method steps in accordance with embodiments disclosed herein.

FIGS. 4A-4B—FIG. 4A is an illustration of a plate map showing position of samples, controls and replicates in a 96-well plate. FIG. 4B shows a graph of the change in OD₅₅₀ after 48-hour exposure as indication of biofilm growth during exposure of Candida tropicalis ATCC 48138 to Tea tree-extracts FIGS. 5A-5B—FIGS. 5A and 5B illustrates a graph of results of anti-biofilm efficacy test against Candida tropicalis ATCC 48138 and Pseudomonas fragi ATCC 4973 (respectively) using Microtiter Dish Biofilm Formation Assay in 96-well plates utilizing a TECAN Fluent high-throughput liquid handling system.

### DETAILED DESCRIPTION

Disclosed herein are methods, systems and devices for screening compounds for efficacy against biofilm formation. These methods have the advantage over the prior art, in that the biofilm formation (or inhibition thereof) is tested directly on the material of interest, thereby providing a high throughput system for testing antimicrobial formulations against corrosion or other consequences of biofilm formation on surfaces of interest.

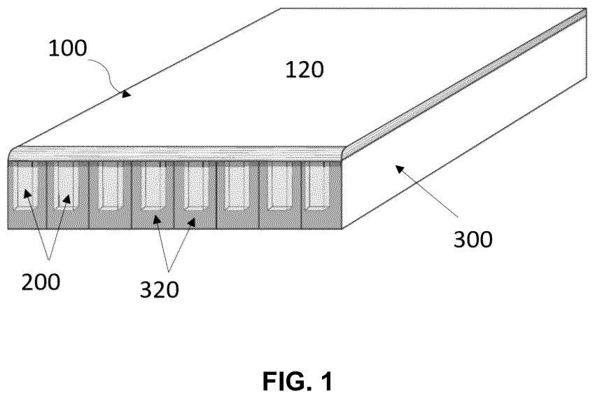

FIG. 1 illustrates a peg assembly 100, in accordance with one embodiment disclosed herein. The peg assembly includes a top surface 120. On the opposite side of the top surface 120, the peg assembly 100 incorporates a plurality downwardly protruding pegs 200. The number of pegs included in the peg assembly can be any specified number. For example, in one embodiment the number of pegs would correspond to standard laboratory well plates, (e.g. 6, 12, 48, 96 pegs) or any customized number. A standard well plate or trough plate 300 is shown in FIG. 1.

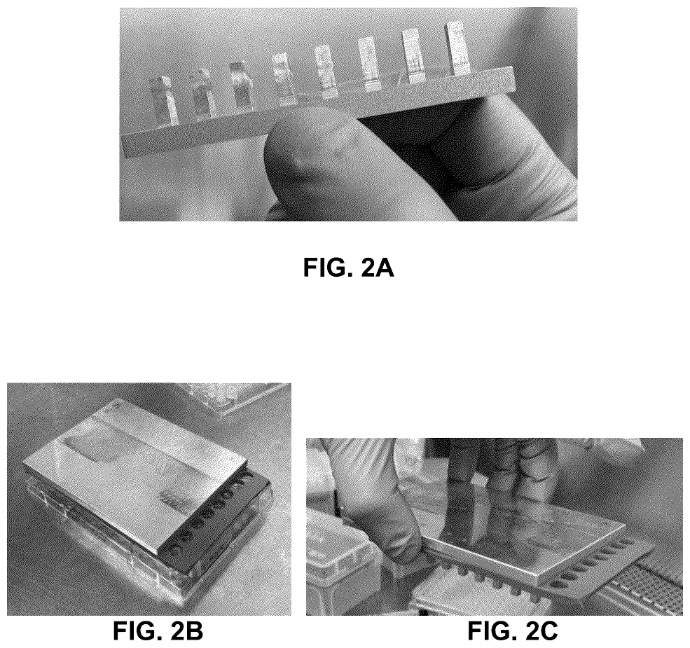

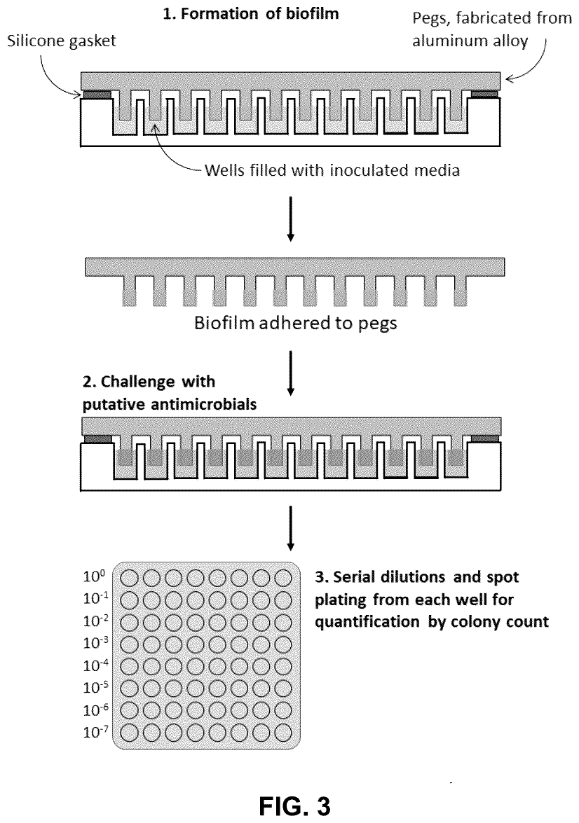

In FIG. 2A, an example of the peg assembly is shown having 8 pegs. In FIGS. 2B and 2C a larger peg assembly is shown having 88 pegs. The pegs shown in FIG. 2A are part of the assemblies shown in FIG. 2B, but are disconnected and separately removable for purposes of experimental controls. This embodiment is one example of a flexible overall strategy in which rows can be removed before the end of the overall incubation for experimental controls or other purposes.

The peg assembly 100 disclosed herein is composed of a material of interest. As used herein, the term “material of interest” refers to the material on which biofilm formation or inhibition is to be tested upon. For example, in one embodiment it will be desirable to test corrosion inhibition on an aluminum alloy that is commonly used in airline fuel tanks (where corrosion is a common problem). The peg assembly will be composed of that specific aluminum alloy so that the corrosion inhibition of certain anti-microbial formulations can be screened/tested directly on the material of interest (instead of a polymeric material which is commonly used in laboratory well peg plates for MBEC assays).

The shape of the pegs can be rectangular, as shown in the embodiments of FIG. 1 and FIGS. 2A-2C. The shape of the pegs can also be cylindrical, conical, or any other desired shape which has a configuration/scale to fit in a standard laboratory well plate 300. The well plate 300 in FIG. 1 allows for insertion of pegs 200 into wells 320. The wells 320 contain inoculated media (potentially composed of relevant industrial fluids like fuel) which can assist with biofilm formation onto the pegs 200.

In one embodiment, the material of interest comprises metallic materials and/or alloys thereof. In one embodiment, the material of interest can be stainless steel, nickel, aluminum, titanium, alloys thereof, or a combination thereof. In certain embodiments, biofilm formation is evaluated on any surface of interest, such as concrete or ceramics. In one embodiment, the peg assembly 100 is comprised of 6061-aluminum alloy.

In other embodiments the pegs 220 can be provided with a with a coating of interest, meaning the corrosion-inhibiting coating to be tested against biofilm formation. Coatings can include zinc oxide, chromate phosphates, manganese and zinc phosphates, fluoropolymers, molybdenum disulfide, epoxy, epoxy phenolic resin, ferrous metal, tetrafluoroethylene (PTFE), polyphenylene sulfide (PPS), or coating formulations with novel bioactive compounds, etc.

Also disclosed herein are methods for screening compounds for efficacy against biofilm formation. In one embodiment the method comprises the steps of:
providing a peg assembly comprised of a material of interest;
growing biofilm on pegs of the peg assembly;
placing the peg assembly onto a challenge plate, wherein the challenge plate comprises at least one compound to be screened; and
evaluating the biofilm remaining on the peg assembly.

As shown in the example of FIG. 3, the peg assembly comprised of a material of interest is provided, and placed into a well plate which contains inoculated media. The inoculated media can contain microbial strains which are to be used in that screening test. Once biofilm formation has occurred on the pegs, the peg assembly is then placed into a challenge plate which contains wells filled with antimicrobial formulations or compounds to be screened. After sufficient exposure to the challenge (for example 6-12 hours), the pegs are rinsed to remove antimicrobial formulations and then sonicated in a well plate filled with PBS buffer, which is subject to dilution. by spot plating for enumeration of viable microorganism remaining on the pegs. Spot plating is the application of small volumes of microbial samples in the challenge plate onto the surface of an agar plate. The challenge plate wells can be filled with serial dilutions of the antimicrobial compounds to be screened/tested. The biofilm remaining on the peg assembly is then evaluated for determination of minimal biofilm eradication concentration (MBEC) by quantification of colony counts.

In one embodiment, the challenge plate wells can contain various compounds to be tested. For example, in a 96-well plate multiple different compounds (and their various concentrations) can be tested at one time on the material of interest, thereby determining in an efficient and short timeframe, which compounds best protect against biofilm formation (and thereby corrosion) for that particular material tested. Control formulations can also be tested as comparison with the antimicrobial compounds screened.

In one embodiment, the methods disclosed herein can be used to simultaneously test multiple antimicrobials from microbial, fungal, plant, or other sources. For example, at least one plant extract to be screened for antimicrobial activity can be derived from cinnamon, clove, garlic, basil, curry ginger sage, oregano, thyme, eucalyptus, ginseng, grapefruit, green tea, peppermint, lavender, rosemary, turmeric, or a combination thereof.

Also disclosed are minimum biofilm eradication concentration (MBEC) assay kits. In one embodiment, the MBEC assay kits include at the following:
at least one peg assembly comprised of a material of interest; and
at least one well plate and/or trough plate.
   The material of interest comprises metallic materials and/or alloys thereof, stainless steel, nickel, aluminum, titanium, or a combination thereof.

The peg assembly disclosed in embodiments above, can be incorporated in the MBEC assay kits discussed herein. In one embodiment, the peg assembly further includes a coating thereon, wherein the coating is incorporated for testing of anti-corrosion properties of the coating.

As shown in FIG. 3, the MBEC assay kit can further incorporate a gasket, comprised for example of silicone. The gasket is intended to cushion the placement of the peg assembly onto the well or trough plate and reduce liquid loss due to evaporation during incubation.

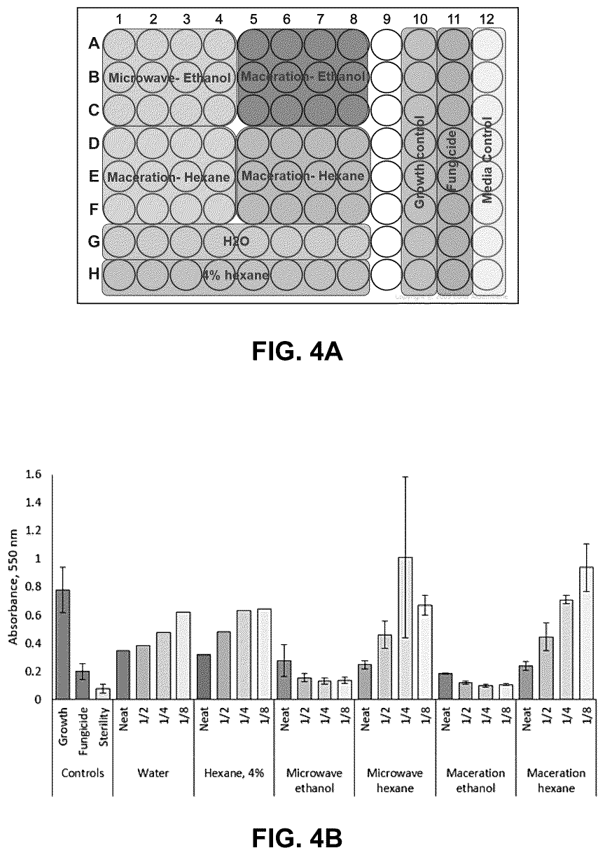

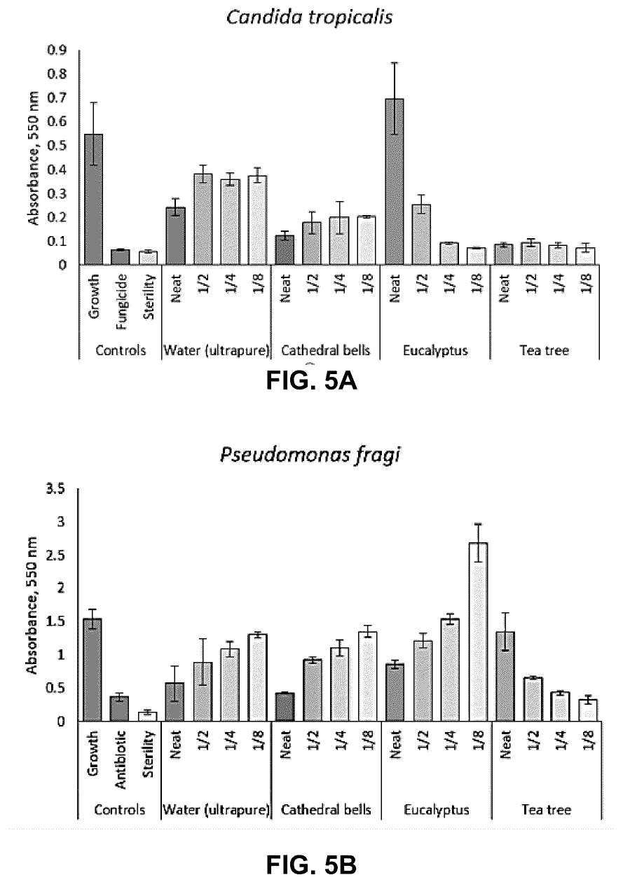

Extracts from Tea tee (Melaleuca alternifolia) plant leaves, prepared using both microwave-assisted extraction and maceration methods, were evaluated for their anti-biofilm efficacy against Candida tropicalis ATCC 48138 using Microtiter Dish Biofilm Formation Assay described by Otoole (2011). The testing was conducted in 96-well plates utilizing a TECAN Fluent high-throughput liquid handling system. Although custom lids with pegs were not used for development of this automated high-throughput method, the custom lid design is easily modified to be suitable for the TECAN Fluent's robot gripper arm (RGA). Extractions were performed using two different solvents: Ethanol (EtOH) and hexane (Hex). Following extraction, the solvent was removed by rotary evaporation at room temperature. Extracts were re-dissolved in sterile distilled H2O for ethanol-based extracts and in 4% hexane for hexane-based ones. The plate map given in FIG. 4A shows the location of the samples and controls. The results demonstrated in FIG. 4B illustrate the varying effects of Tea Tree leaves-extract (neat) and its dilutions (½, ¼ and ⅛) on biofilm formation, as indicated by OD₅₅₀ nm readings after 48h-exposure time (at 28° C.). Positive control demonstrates robust growth of Candida tropicalis ATCC 48138 (growth control), while the Fungicide (Amphotericin B, final concentration in well as 12.5 μg/mL), media control (Sterility control, Luria-Bertani broth), H₂O and 4% hexane (the diluents used for re-suspension of the extracts, added in the same volumes as the “neat,” “½,” “¼,” and “⅛” extracts) controls confirm the assay's baseline and the efficacy of the inhibitory control. Among the test compounds, the Mace-EtOH (Maceration extraction, extraction solvent: Ethanol) series consistently exhibited very low OD₅₅₀ readings, suggesting a strong inhibitory effect akin to the fungicide, even at diluted concentrations. In contrast, the Mace-Hex (Maceration extraction, extraction solvent: Hexane) series showed a dose-dependent increase in OD₅₅₀ values at higher dilutions, indicating a diminishing inhibitory effect, with Mace-Hex with ⅛-dilution showing the highest OD₅₅₀ within this group. The Micro-EtOH (Microwave-assisted extraction, extraction solvent: Ethanol) and Micro-Hex (Microwave-assisted extraction, extraction solvent: Hexane) series present more nuanced responses, generally showing a decrease in OD with increasing dilution, but with Micro-Hex notably demonstrated increase in OD values (except for Micro-Hex-⅛), like Mace-Hex series. Overall, ethanol extracts of Tea Tree obtained from both microwave-assisted extraction and maceration methods demonstrated a significant antimicrobial effect against Candida tropicalis ATCC 48138 biofilms at neat and its diluted counterparts (½, ¼, 1/8 dilutions), outperforming both the positive and other controls and hexane-based extracts.

FIG. 5A shows results of anti-biofilm efficacy test against Candida tropicalis ATCC 48138 using Microtiter Dish Biofilm Formation Assay in 96-well plates utilizing a TECAN Fluent high-throughput liquid handling system. The plate map (top) shows the position of samples, controls and replicates positioned in the 96-well plate. The bar graph shows the change in OD₅₅₀ after 48h-exposure as indication of biofilm growth during exposure of Candida tropicalis ATCC 48138 to Tea tree-extracts obtained with microwave-assisted and maceration extraction methods.

The extracts from Cathedral bells (Kalanchoe pinnata), Eucalyptus (Eucalyptus globulus), and Tea tree were prepared using a microwave-assisted method and performed a Microtiter Dish Biofilm Formation Assay in 96-well plate to assess the extracts' impact on biofilm formation against Candida tropicalis ATCC 48138. The TECAN Fluent system was used to automate the process. Extracts of Cathedral bells at different concentrations (neat, dilutions of 1:2, 1:4, 1:8) show relatively low OD₅₅₀ values, similar to the fungicide (Amphotericin B, final concentration in well as 12.5 μg/mL) and sterility (Luria-Bertani broth) controls, suggesting that Cathedral bells extract significantly inhibits the growth of C. tropicalis after 48h-exposure at 28° C. As concentration decreases (1:2, 1:4, 1:8), there appears to be a slight increase in OD₅₅₀, suggesting a dose-dependent effect where lower concentrations are less inhibitory. The data for Eucalyptus extracts (FIG. 5B) shows a very high OD₅₅₀ value, similar to the growth control, indicating that at this concentration, Eucalyptus promotes C. tropicalis growth. However, as the concentration decreases (dilution ratios of 1:2, 1:4, 1:8), the OD₅₅₀ values significantly drop, suggesting that lower concentrations of Eucalyptus extracts are less conducive to C. tropicalis growth, or perhaps even inhibitory at very high dilutions not shown, but at the initial concentration, it seems to enhance growth. All tested concentrations of Tea Tree extracts (neat, 1:2, 1:4, 1:8) consistently show low OD₅₅₀ values, comparable to the sterility and fungicide controls, indicating that Tea Tree extracts effectively inhibit the growth of C. tropicalis across these concentrations. These results are also consistent with the results demonstrated in FIG. 4B for Tea Tree extracts. Overall, Cathedral bells and Tea Tree extracts exhibit antifungal properties against C. tropicalis, as indicated by the low OD₅₅₀ values, suggesting inhibition of fungal growth. The observation of consistent results for Tea Tree extracts, despite the tests being conducted on different days (FIG. 5A and FIG. 5B), demonstrates that the developed high-throughput screening method in 96-well plate to assess the anti-biofilm effects of extracts/compounds yields reliable and reproducible outcomes.

The extracts from Cathedral bells, Eucalyptus, and Tea tree (extracted via the microwave-assisted method) were tested this time to assess their impact on biofilm formation against Pseudomonas fragi ATCC 4973 using the developed high-throughput screening system. Among the control groups, the growth control showed the highest absorbance, indicating active bacterial proliferation, while the antibiotic control (Kanamycin, 50 μg/mL) and sterility control (Nutrient broth media) had significantly lower OD₅₅₀ values, confirming inhibition and absence of growth, respectively. Ultrapure water control across all dilutions (Neat to ⅛) maintained relatively high absorbance, suggesting minimal antimicrobial activity. Cathedral Bells extract demonstrated a dose-dependent reduction in absorbance, with the Neat and 1/2 dilutions showing noticeable inhibition, indicating potential antimicrobial properties. Eucalyptus extract followed a similar trend, with reduced absorbance at higher concentrations, supporting its effectiveness against Pseudomonas fragi. Notably, Tea Tree extract consistently showed low absorbance across all dilutions, even on different testing days, highlighting its strong and reproducible antimicrobial activity. These results collectively validate the reliability of the high-throughput screening method and underscore the potential of plant extracts, particularly Tea Tree, in microbial inhibition.

Using a high-throughput screening (HTS) method with peg-based system to evaluate the antimicrobial/anti-biofilm activity of extracts is essential for efficiently identifying promising candidates from a large pool of natural substances. HTS with 96-well peg format allows for rapid, parallel testing of multiple samples under standardized conditions, ensuring consistency and reproducibility across experiments. This approach not only accelerates the discovery process but also minimizes variability and human error, making it ideal for comparing the efficacy of different extracts and dilutions. The multi-well/peg format via HTS enables a library of compounds to be screened against biofilms adhered to the pegs, which enable specific microbial adhesion mechanisms, redox interactions, corrosion mechanisms, surface properties of the material of interest. Also, it provides a robust platform to detect subtle differences in activity, optimize concentrations, and prioritize extracts for further investigation, ultimately supporting the development of effective, antimicrobial/anti-biofilm agents.

Those skilled in the art will understand from the foregoing description that modifications and changes may be made in various embodiments of the present disclosure without departing from its true spirit of the invention. The descriptions in this specification are for purposes of illustration only and are not to be construed in a limiting sense.

### REFERENCES

Hage, M., et al., Antimicrobial peptides-coated stainless steel for fighting biofilms formation for food and medical fields: Review of literature. Coatings, 2021. 11(10): p. 1216.
2. Ceri, H., et al., The Calgary Biofilm Device: new technology for rapid determination of antibiotic susceptibilities of bacterial biofilms. Journal of clinical microbiology, 1999. 37(6): p. 1771-1776.
3. Mohr, K. I., et al., Pinensins: the first antifungal lantibiotics. Angewandte Chemie International Edition, 2015. 54(38): p. 11254-11258.
4. Karpiński, T. M., et al., Anti-Candida and Antibiofilm Activity of Selected Lamiaceae Essential Oils. Frontiers in Bioscience-Landmark, 2023. 28(2): p. 28.
5. Itah, A., et al., Biodegradation of international jet A-1 aviation fuel by microorganisms isolated from aircraft tank and joint hydrant storage systems. Bulletin of environmental contamination and toxicology, 2009. 83: p. 318-327.
6. McNamara, C. J., et al., Corrosion of aluminum alloy 2024 by microorganisms isolated from aircraft fuel tanks. Biofouling, 2005. 21(5-6): p. 257-265.
7. Striebich, R. C., et al., Characterization of the F-76 diesel and Jet-A aviation fuel hydrocarbon degradation profiles of Pseudomonas aeruginosa and Marinobacter hydrocarbonoclasticus. International Biodeterioration & Biodegradation, 2014. 93: p. 33-43.
8. ASTM E2799-12: Testing Disinfectant Efficacy against Pseudomonas aeruginosa Biofilm using the MBEC Assay. ASTM.
9. Jovanović, A. A., et al., The influence of different extraction conditions on polyphenol content, antioxidant and antimicrobial activities of wild thyme. Journal of Applied Research on Medicinal and Aromatic Plants, 2021. 25: p. 100328

What is claimed is:

1. A method for screening compounds for efficacy against biofilm formation, the method comprising:
providing a peg assembly comprised of a material of interest;
growing biofilm on pegs of the peg assembly;
placing the peg assembly onto a challenge plate, wherein the challenge plate comprises at least one compound to be screened; and
evaluating the biofilm remaining on the peg assembly.

2. The method of claim 1, wherein the material of interest are selected from the group consisting of metals or metal alloys, stainless steel, nickel, aluminum, titanium, concrete, ceramics, polymers or a combination thereof.

3. The method of claim 1, wherein the material of interest further comprises a coating thereon to be tested for corrosion protection.

4. The method of claim 1, wherein the step of growing a biofilm on pegs of the peg assembly comprises placing the peg assembly into an inoculation plate containing inoculated media.

5. The method of claim 1, wherein the at least one compound to be screened comprises an antimicrobial compound containing at least one plant extract.

6. The method of claim 5, wherein the at least one plant extract is derived from cinnamon, clove, garlic, basil, curry ginger sage, oregano, thyme, eucalyptus, ginseng, grapefruit, green tea, peppermint, lavender, rosemary, turmeric, or a combination thereof.

7. The method of claim 1, wherein the challenge plate contains serial dilutions of the at least one compound to be screened.

8. The method of claim 1, wherein the step of evaluating the biofilm remaining on the peg assembly comprises determining minimal biofilm eradication concentration (MBEC) through quantitative analysis of microbial colony counts.

9. The method of claim 1, wherein the peg assembly comprises plurality of pegs comprised from an aluminum alloy.

10. The method of claim 1, wherein the screened compounds are plant extracts screened for antimicrobial activity.

11. A minimum biofilm eradication concentration (MBEC) assay kit, comprising:
at least one peg assembly comprised of a material of interest; and
at least one well plate and/or trough plate;
wherein the material of interest comprises metallic materials and/or alloys thereof, stainless steel, nickel, aluminum, titanium, concrete, ceramics, polymers, or a combination thereof.

12. The kit of claim 11, wherein the peg assembly comprises a plurality of pegs.

13. The kit of claim 11, wherein the peg assembly further comprises a coating thereon, wherein the coating is incorporated for testing of anti-corrosion properties of the coating.

14. The kit of claim 11, wherein the peg assembly comprises square pegs protruding from a bottom side.

15. The kit of claim 11, wherein the kit further comprises a gasket.

16. The kit of claim 11, wherein the peg assembly comprises a plurality of pegs, wherein the number of pegs corresponds to standardized laboratory well plates.

17. A method for screening compounds for efficacy against biofilm formation, the method comprising:
i. providing a peg assembly comprised of a material of interest;
ii. growing biofilm on pegs of the peg assembly;
iii. placing the peg assembly onto a challenge plate, wherein the challenge plate comprises serial dilutions of the at least one compound to be screened; and
iv. evaluating the biofilm remaining on the peg assembly.

18. The method of claim 17, further comprising the step of coating the peg assembly with a coating for testing of anti-corrosion properties of the coating.

19. The method of claim 17, wherein the screened compounds are plant extracts screened for antimicrobial activity.

20. The method of claim 17, wherein the material of interest comprises metallic materials and/or alloys thereof, stainless steel, nickel, aluminum, titanium, concrete, ceramics, polymers, or a combination thereof
